(19) United States
(12) Patent Application Publication
(10) Pub. No.: US 2024/0408335 A1
(43) Pub. Date: Dec. 12, 2024

(54) EXACERBATION RISK PREDICTION SYSTEM

(71) Applicant: DAIKIN INDUSTRIES, LTD., Osaka (JP)

(72) Inventors: Takahiro HIRAYAMA, Osaka (JP); Masahiro KATO, Osaka (JP); Kenji HAYASHI, Osaka (JP); Tomoya HIRANO, Osaka (JP); Satoshi HASHIMOTO, Osaka (JP)

(21) Appl. No.: 18/809,037

(22) Filed: Aug. 19, 2024

Related U.S. Application Data
(63) Continuation of application No. PCT/JP2023/007256, filed on Feb. 28, 2023.

(30) Foreign Application Priority Data
Mar. 4, 2022 (JP) 2022-033620

(51) Int. Cl. A61M 16/00; A61M 16/10; G16H 50/20; G16H 50/30

(52) U.S. Cl. CPC A61M 16/026; A61M 16/101; G16H 50/20; G16H 50/30; A61M 2230/42

(57) ABSTRACT

An exacerbation risk prediction system predicts an exacerbation risk of a patient suffering from a respiratory disease. The exacerbation risk prediction system includes an oxygen concentrator that supplies oxygen to the patient through a cannula, an acquisition unit that acquires first information including biological information of the patient wearing the cannula on exertion and at rest and patient information regarding a disease state, a storage that stores the first information, a prediction unit that predicts the exacerbation risk based on the first information, and a learning unit that learns the first information and an evaluation regarding exacerbation in association with each other. The biological information is at least one of respiratory rate, respiratory waveform, and exhaled gas component amount. The biological information is acquired from the oxygen concentrator. The prediction unit predicts the exacerbation risk by inputting the first information to a learning model created by the learning unit.

### CROSS-REFERENCE TO RELATED APPLICATIONS

This is a continuation of International Application No. PCT/JP2023/007256 filed on Feb. 28, 2023, which claims priority to Japanese Patent Application No. 2022-033620, filed on Mar. 4, 2022. The entire disclosures of these applications are incorporated by reference herein.

### BACKGROUND

### Technical Field

The present disclosure relates to an exacerbation risk prediction system.

### Background Art

A patient suffering from a respiratory disease such as COPD (chronic obstructive pulmonary disease) will be hospitalized when exacerbation occurs, and medical costs increase. In order to suppress the exacerbation, it is necessary to predict an exacerbation risk and to take measures in advance so that the exacerbation does not occur. JP 7000423 B2 discloses a technique of predicting the exacerbation risk based on biological information of a patient.

### SUMMARY

An exacerbation risk prediction system according to a first aspect predicts an exacerbation risk of a patient suffering from a respiratory disease. The exacerbation risk prediction system includes an acquisition unit, a storage, and a prediction unit. The acquisition unit acquires first information including biological information and patient information. The biological information is information of the patient on exertion and at rest. The patient information is information of the patient regarding a disease state. The storage stores the first information. The prediction unit predicts the exacerbation risk based on the first information.

### BRIEF DESCRIPTION OF THE DRAWINGS

FIG. 1 is a schematic configuration diagram of an exacerbation risk prediction system.

FIG. 2 is a functional block diagram of a prediction device.

FIG. 3 is a functional block diagram of an oxygen concentrator.

FIG. 4 is a flowchart for describing an example of processing of the exacerbation risk prediction system.

### DETAILED DESCRIPTION OF EMBODIMENT(S)

### (1) Overall Configuration

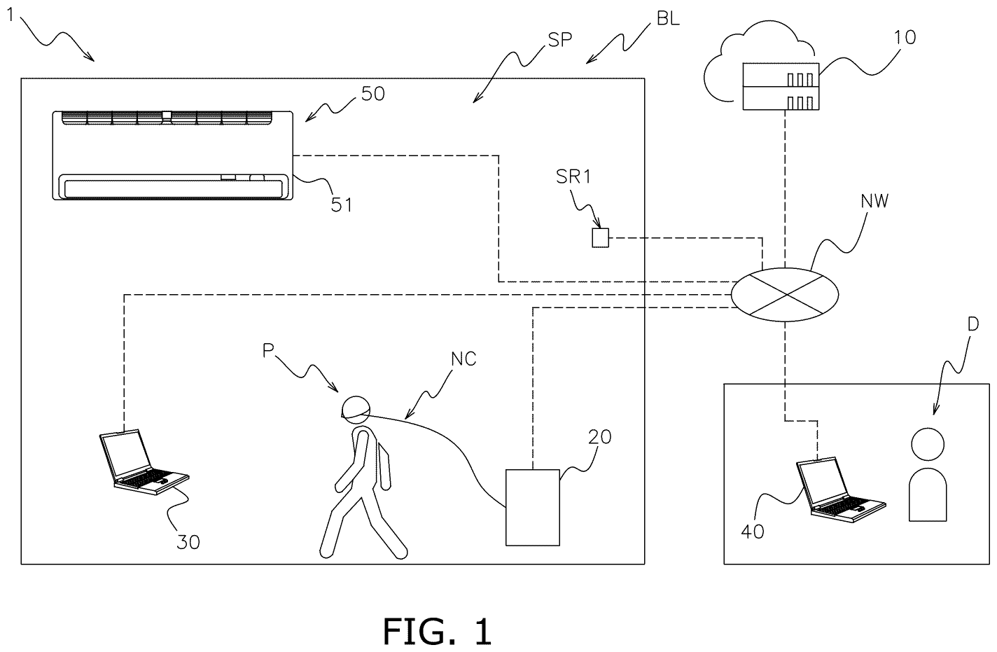

An exacerbation risk prediction system 1 predicts an exacerbation risk of a patient P suffering from a respiratory disease. FIG. 1 is a schematic configuration diagram of the exacerbation risk prediction system 1. As illustrated in FIG. 1, the exacerbation risk prediction system 1 includes a prediction device 10, an oxygen concentrator 20, a patient terminal 30, a doctor terminal 40, and an air conditioner 50. The prediction device 10, the oxygen concentrator 20, the patient terminal 30, the doctor terminal 40, and the air conditioner 50 are communicably connected via a network NW. Examples of the network NW include the Internet.

A stationary oxygen concentrator 20 is installed in a target space SP inside a building BL. The oxygen concentrator 20 supplies oxygen to the patient P through a cannula NC. While wearing the cannula NC, the patient P is moving around inside the building BL or resting inside the building BL. The patient P inputs various types of information such as patient information D2 (described later) to the prediction device 10 via the patient terminal 30.

The air conditioner 50 for performing air conditioning of the target space SP is installed in the building BL. In particular, an indoor unit 51 is installed in the target space SP. In addition, an indoor environment sensor SR1 such as a temperature sensor and a humidity sensor is installed in the target space SP. The prediction device 10 and the indoor environment sensor SR1 are communicably connected via the network NW.

The patient P regularly (in the present embodiment, once a month) visits a hospital and receives an examination of a doctor D. The doctor D inputs a diagnosis result or the like at a time of examination to the prediction device 10 via the doctor terminal 40.

The prediction device 10 acquires various types of information from the oxygen concentrator 20, the patient terminal 30, the doctor terminal 40, and the indoor environment sensor SR1, and predicts the exacerbation risk of the patient P. The prediction device 10 transmits various types of information to the oxygen concentrator 20 and the air conditioner 50 based on the predicted exacerbation risk of the patient P.

### (2) Detailed Configuration

### (2-1) Prediction Device

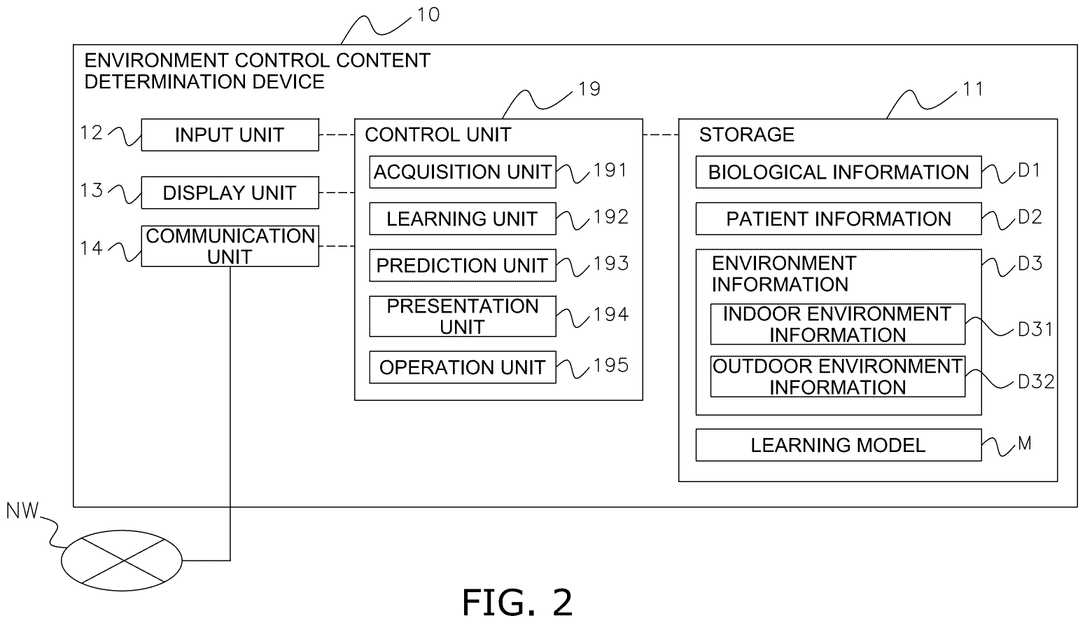

The prediction device 10 is a computer installed on a cloud. FIG. 2 is a functional block diagram of the prediction device 10. As illustrated in FIG. 2, the prediction device 10 mainly includes a storage 11, an input unit 12, a display unit 13, a communication unit 14, and a control unit 19.

### (2-1-1) Storage

The storage 11 is a storage device such as a RAM, a ROM, and an HDD. The storage 11 stores a program implemented by the control unit 19, data necessary for implementing the program, and the like. In the present embodiment, the storage 11 particularly stores first information to be described later.

### (2-1-2) Input Unit

The input unit 12 is a keyboard and a mouse. Various commands and various types of information for the prediction device 10 can be input by using the input unit 12.

### (2-1-3) Display Unit

The display unit 13 is a monitor. The display unit 13 can display various types of information and the like stored in the storage 11.

### (2-1-4) Communication Unit

The communication unit 14 is a network interface device for communicating with the oxygen concentrator 20 and the like via the network NW.

### (2-1-5) Control Unit

The control unit 19 is a processor such as a CPU or a GPU. The control unit 19 reads and executes the program stored in the storage 11 to implement various functions of the prediction device 10. The control unit 19 can also write a calculation result to the storage 11 and read information stored in the storage 11 in accordance with the program.

As illustrated in FIG. 2, the control unit 19 mainly includes an acquisition unit 191, a learning unit 192, a prediction unit 193, a presentation unit 194, and an operation unit 195.

### (2-1-5-1) Acquisition Unit

The acquisition unit 191 acquires first information including biological information D1, patient information D2, and environment information D3. The first information is information serving as an explanatory variable of a learning model M to be described later. In the present embodiment, the first information includes the biological information D1, the patient information D2, and the environment information D3.

### (a) Biological Information

The biological information D1 is information of the patient P on exertion and at rest. The biological information D1 is at least one of a respiratory rate, a respiratory waveform, an exhaled gas component amount, a blood oxygen concentration, a heart rate, a height, or a weight. The respiratory rate is the number of breaths per minute. The respiratory waveform is a waveform in which the intensities of inspiration and expiration within a predetermined time are expressed as positive and negative amplitudes. In other words, the respiratory waveform is a value of amplitude at a plurality of times within a predetermined time. The exhaled gas component amount is a ratio of each component such as oxygen and carbon dioxide contained in the exhaled gas.

In the present embodiment, the respiratory rate, the respiratory waveform, and the exhaled gas component amount are used as the biological information D1. The acquisition unit 191 acquires the respiratory rate, the respiratory waveform, and the exhaled gas component amount from the oxygen concentrator 20 every minute.

Note that, in a case where the blood oxygen concentration and the heart rate are used as the biological information D1, the acquisition unit 191 acquires the blood oxygen concentration and the heart rate from, for example, a pulse oximeter or a smart watch worn by the patient P every minute. At this time, the patient P wears the pulse oximeter or the smart watch in advance. Furthermore, in a case where the height and the weight are used as the biological information D1, the acquisition unit 191 acquires the height and the weight from the patient P via the patient terminal 30 once a month, for example.

### (b) Patient Information

The patient information D2 is information of the patient P regarding a disease state. The patient information D2 is at least one of a degree of progress of the disease state, a forced expiratory volume versus standard, a prescribed flow rate, a medical history, the type of medicine being taken, a degree of cough, a degree of sputum, a color of sputum, a degree of shortness of breath, a degree of sleep, or a physical condition. The degree of progress of the disease state is a value from “stage I” to “stage IV” indicating a stage of the disease state. The forced expiratory volume versus standard is a percentage value with respect to a predicted forced expiratory volume by age, body type, and sex. The prescribed flow rate is an oxygen flow rate on exertion and at rest prescribed by the doctor D and supplied from the oxygen concentrator 20 to the patient P. The medical history is a disease name such as “diabetes”. The type of medicine being taken is the name of the medicine being taken. The degree of cough, the degree of sputum, the degree of shortness of breath, the degree of sleep, and the physical condition are represented by, for example, ten numerical values indicating the degrees. The color of sputum is represented by a color name such as “yellowish green” and “green”.

In the present embodiment, as the patient information D2, the degree of progress of the disease state, the forced expiratory volume versus standard, the degree of cough, the degree of sputum, the color of sputum, the degree of shortness of breath, the degree of sleep, and the physical condition are used. The acquisition unit 191 acquires the degree of progress of the disease state and the forced expiratory volume versus standard from the doctor D via the doctor terminal 40 at the time of the monthly examination. In addition, the acquisition unit 191 acquires the degree of cough, the degree of sputum, the color of sputum, the degree of shortness of breath, the degree of sleep, and the physical condition from the patient P via the patient terminal 30 once a month.

Note that, in a case where the prescribed flow rate, the medical history, and the type of medicine being taken are used as the patient information D2, the acquisition unit 191 acquires the prescribed flow rate, the medical history, and the type of medicine being taken from the doctor D via the doctor terminal 40 at the time of the monthly examination, for example.

### (c) Environment Information

The environment information D3 is information of the indoor and/or outdoor of the building BL in which the patient P is present. The environment information D3 of an indoor of the building BL (hereinafter, the indoor environment information may be referred to as indoor environment information D31) is at least one of temperature, humidity, carbon dioxide concentration, carbon monoxide concentration, ozone concentration, sulfur trioxide (SO3) concentration, temperature difference from the outdoor, dust amount, PM2.5 amount, yellow sand amount, mold amount, virus amount, volatile organic compounds (VOC) amount, pollen amount, allergic substance amount, bacteria amount, oxygen concentration, airflow, or atmospheric pressure. The environment information D3 of an outdoor of the building BL (hereinafter, the outdoor environment information may be referred to as outdoor environment information D32) is at least one of temperature, humidity, weather, atmospheric pressure, dust amount, PM2.5 amount, or yellow sand amount.

In the present embodiment, the temperature and humidity are used as the indoor environment information D31. The acquisition unit 191 acquires the temperature and the humidity of the target space SP from the indoor environment sensor SR1 (specifically, a temperature sensor and a humidity sensor) every minute. In the present embodiment, the temperature and the humidity are used as the outdoor environment information D32. The acquisition unit 191 acquires outdoor temperature and humidity from an external server of the Japan Meteorological Agency or the like via the network NW every minute.

In a case where the carbon dioxide concentration, the carbon monoxide concentration, the ozone concentration, the SO3 concentration, the temperature difference from the outdoor, the dust amount, the PM2.5 amount, the yellow sand amount, the mold amount, the virus amount, the VOC amount, the pollen amount, the allergic substance amount, the bacteria amount, the oxygen concentration, the airflow, and the atmospheric pressure are used as the indoor environment information D31, the acquisition unit 191 acquires these pieces of information from the indoor environment sensor SR1 capable of measuring each piece of information, for example, every minute. When the weather, the atmospheric pressure, the dust amount, the PM2.5 amount, and the yellow sand amount are used as the outdoor environment information D32, the acquisition unit 191 acquires these pieces of information from an external server of the Japan Meteorological Agency or the like via the network NW, for example, every minute.

### (2-1-5-2) Learning Unit

The learning unit 192 learns the biological information D1, the patient information D2, and the environment information D3 (first information) in association with an evaluation regarding exacerbation.

The learning unit 192 creates a learning data set for creating a learning model M having the biological information D1, the patient information D2, and the environment information D3 as explanatory variables and the evaluation regarding exacerbation as an objective variable. In the present embodiment, the learning unit 192 creates one record of learning data set every minute (hereinafter, the record of the learning data set may be described as a learning record). The biological information D1, the patient information D2, and the environment information D3 of the newly created learning record are the biological information D1, the patient information D2, and the environment information D3 acquired by the acquisition unit 191 immediately before. For example, since the “respiratory rate” included in the biological information D1 is acquired every minute, the value of the “respiratory rate” of learning data can be different for each learning record. Furthermore, for example, since the “degree of progress of the disease state” included in the patient information D2 is acquired once a month, the value of the “degree of progress of the disease state” of the learning data set can change about every month.

The evaluation regarding exacerbation takes a binary value of “exacerbation (=1)” or “no exacerbation (=0)”. Every time a learning record is created, the learning unit 192 sets an evaluation regarding exacerbation based on the biological information D1, the patient information D2, and the environment information D3 of the learning record. For example, when all the information such as “the degree of progress of the disease state” included in the patient information D2 exceeds each threshold value stored in advance, the learning unit 192 sets the evaluation regarding exacerbation to “exacerbation”. Furthermore, for example, the learning unit 192 compares the “respiratory waveform” included in the biological information D1 with the respiratory waveform at the time of exacerbation stored in advance, and when the difference between the respiratory waveforms exceeds a predetermined value, the learning unit 192 sets the evaluation regarding exacerbation to “exacerbation”.

The learning unit 192 creates the learning model M by using the learning data set. In the present embodiment, the learning unit 192 creates the learning model M once a week. When creating the learning model M for the first time, the learning unit 192 creates the learning model M by using the learning data set created so far. When creating the learning model M for the second time or later, the learning unit 192 creates the learning model M by using the learning data set created after the time of the previous creation of the learning model M (which means updating of the learning model M in this case). In the present embodiment, the learning model M is a fully connected neural network. However, the learning model M is not limited to this network, and other learning models such as SVM, random forest, and XGBoost may be used. The learning model M has nodes of the number of explanatory variables in an input layer and one node in an output layer. An activation function of an intermediate layer of the learning model M is a ramp function, and an activation function of the output layer of the learning model M is a sigmoid function. In other words, when the biological information D1, the patient information D2, and the environment information D3 are input, the learning model M outputs a real value from “0” to “1” indicating a probability that the evaluation regarding exacerbation is “exacerbation”.

In addition, the learning unit 192 determines, from the indoor environment information D31, an explanatory variable that most contributes to the “evaluation regarding exacerbation” (which may be hereinafter described as a contribution variable) and a target value of a contribution variable in which the value of the “evaluation regarding exacerbation” to be output can be smaller than a predetermined value (in the present embodiment, “0.5”). The learning unit 192 determines the contribution variable and the target value of the contribution variable by using, for example, a filter method, a wrapper method, or the like. When the learning model M is a random forest or the like, the learning unit 192 may determine the contribution variable and the target value of the contribution variable by using an integration method.

### (2-1-5-3) Prediction Unit

The prediction unit 193 predicts the exacerbation risk based on the biological information D1, the patient information D2, and the environment information D3 (first information). Specifically, the prediction unit 193 predicts the exacerbation risk by inputting the biological information D1, the patient information D2, and the environment information D3 acquired by the acquisition unit 191 to the learning model M created by the learning unit 192.

In the present embodiment, the exacerbation risk is expressed by an integer value from “0” to “10”. The higher the numerical value, the higher the exacerbation risk. The prediction unit 193 inputs the biological information D1, the patient information D2, and the environment information D3 to the learning model M, and outputs a real value from “0” to “1” indicating a probability that the evaluation regarding exacerbation is “exacerbation”. The prediction unit 193 predicts, as the exacerbation risk, a value obtained by rounding down the output value after the second decimal place, and further multiplying the rounded down value by 10.

The prediction unit 193 predicts the exacerbation risk by inputting the biological information D1, the patient information D2, and the environment information D3 acquired immediately before by the acquisition unit 191 every minute to the learning model M. In other words, the prediction unit 193 predicts the exacerbation risk by inputting the biological information D1, the patient information D2, and the environment information D3 of the learning record created every minute to the learning model M.

### (2-1-5-4) Presentation Unit

The presentation unit 194 presents a guide regarding the exacerbation risk to the patient P based on the exacerbation risk predicted by the prediction unit 193. The guide regarding the exacerbation risk will vary depending on the value of the predicted exacerbation risk. When the exacerbation risk is “5”, the guide regarding the exacerbation risk is, for example, text data “(exacerbation risk “5”) caution: take rest”. When the exacerbation risk is “8”, the guide regarding the exacerbation risk is, for example, text data “(exacerbation risk “8”) warning: recommended to consult a medical institution”.

In the present embodiment, the presentation unit 194 presents the guide regarding the exacerbation risk to the patient P via the oxygen concentrator 20. In the present embodiment, when the exacerbation risk is “5” or higher, the presentation unit 194 transmits a guide regarding the exacerbation risk to the oxygen concentrator 20.

### (2-1-5-5) Operation Unit

The operation unit 195 operates the air conditioner 50 installed in the building BL so as to improve the exacerbation risk based on the exacerbation risk predicted by the prediction unit 193. In the present embodiment, when the exacerbation risk predicted by the prediction unit 193 is a predetermined value (in the present embodiment, “5”) or more, the operation unit 195 transmits control information to the air conditioner 50 so that the exacerbation risk predicted next becomes smaller than the predetermined value based on the contribution variable determined by the learning unit 192 and the target value of the contribution variable. For example, when the contribution variable is “temperature” and the target value of the contribution variable is “25° C.”, the operation unit 195 transmits the control information to the air conditioner 50 so that the temperature of the target space SP becomes 25° C.

### (2-2) Oxygen Concentrator

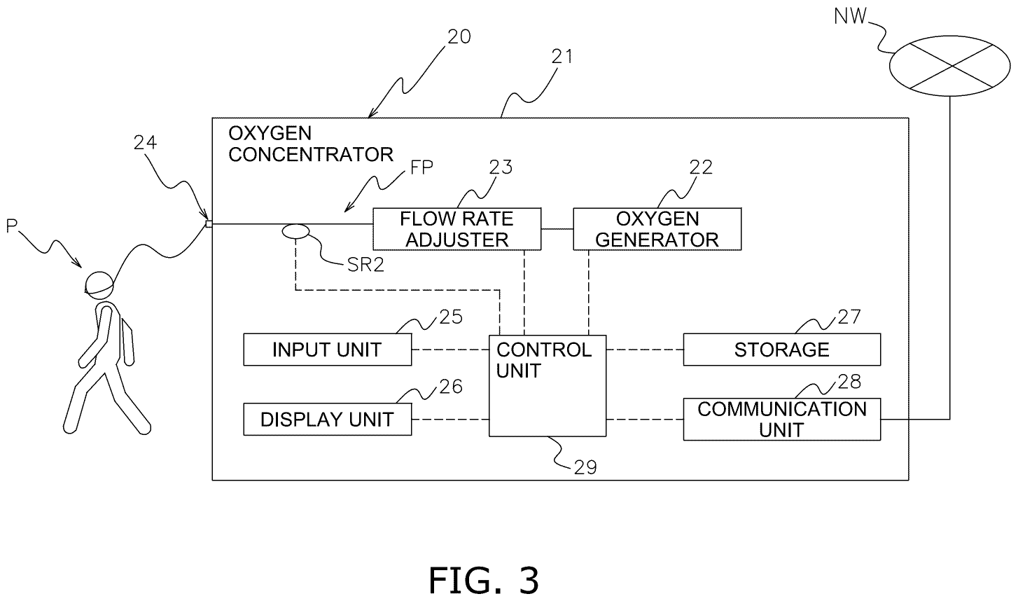

FIG. 3 is a functional block diagram of the oxygen concentrator 20. As illustrated in FIG. 3, the oxygen concentrator 20 includes a casing 21, an oxygen generator 22, a flow rate adjuster 23, a storage 27, a communication unit 28, and a control unit 29 installed inside the casing 21, and an oxygen discharger 24, an input unit 25, and a display unit 26 installed on a surface of the casing 21.

The oxygen generator 22 generates an oxygen concentrated gas. The oxygen generator 22 and the oxygen discharger 24 are connected by a flow path FP via the flow rate adjuster 23. The cannula NC is attached to the oxygen discharger 24. A pair of attachment portions to be attached to the nose of the patient P is provided at a distal end of the cannula NC on the patient P side. Oxygen generated by the oxygen generator 22 is supplied to the patient P via the flow path FP, the oxygen discharger 24, and the cannula NC. At this time, the flow rate adjuster 23 adjusts an oxygen flow rate to be supplied to the patient P.

The input unit 25 is a device for inputting an oxygen flow rate or the like prescribed by the doctor D. The display unit 26 is a screen for displaying the current oxygen flow rate and the like. The storage 27 is a storage device such as a RAM, a ROM, or an HDD. The communication unit 28 is a network interface device for communicating with the prediction device 10 and the like via the network NW.

A sensor SR2 for detecting a respiratory rate, a respiratory waveform, and an exhaled gas component amount is provided in the flow path FP between the flow rate adjuster 23 and the oxygen discharger 24.

The control unit 29 is a processor such as a CPU or a GPU. The control unit 29 reads and executes a program stored in the storage 27 to implement various functions of the oxygen concentrator 20. The control unit 29 can also write a calculation result to the storage 27 and read information stored in the storage 27 in accordance with the program.

For example, the control unit 29 controls the flow rate adjuster 23 based on the oxygen flow rate input from the input unit 25 to adjust the oxygen flow rate to be supplied to the patient P. In addition, for example, the control unit 29 detects the respiratory rate, the respiratory waveform, and the exhaled gas component amount by the sensor SR2 every minute, and transmits these pieces of information to the prediction device 10. Furthermore, for example, when receiving the guide regarding the exacerbation risk from the prediction device 10, the control unit 29 displays the guide regarding the exacerbation risk on the display unit 26. At this time, the control unit 29 may be configured to display the guide regarding the exacerbation risk together with a voice.

### (2-3) Patient Terminal and Doctor Terminal

The patient terminal 30 and the doctor terminal 40 are general personal computers. The patient terminal 30 and the doctor terminal 40 include a CPU and a storage device such as a RAM, a ROM, or an HDD. In addition, the patient terminal 30 and the doctor terminal 40 include a communication unit for connecting to the network NW. The patient terminal 30 and the doctor terminal 40 include a keyboard and a mouse as information input units. The patient terminal 30 and the doctor terminal 40 include a display as an information display unit.

The patient P inputs the biological information D1 and the like to the prediction device 10 via the patient terminal 30 once a month. In addition, the doctor D inputs the patient information D2 and the like to the prediction device 10 via the doctor terminal 40 at the time of the monthly examination.

### (2-4) Air Conditioner

The air conditioner 50 constitutes a vapor compression refrigeration cycle and performs air conditioning of the target space SP. The air conditioner 50 mainly includes the indoor unit 51, an outdoor unit, and a controller. The indoor unit 51 and the outdoor unit constitute a refrigerant circuit. The indoor unit 51 and the outdoor unit are communicably connected by a communication line.

As illustrated in FIG. 1, in the present embodiment, the indoor unit 51 is a wall-mounted unit installed on a wall of the target space SP. The indoor unit 51 mainly includes an indoor heat exchanger, an indoor fan, an indoor control unit, and various sensors such as an indoor temperature sensor. The indoor heat exchanger causes a refrigerant flowing through the indoor heat exchanger to exchange heat with air in the target space SP. The indoor fan is a fan that supplies air in the target space SP to the indoor heat exchanger. The indoor control unit controls the operation of each component constituting the indoor unit 51. The indoor control unit includes a control calculation device and a storage device.

The outdoor unit is installed outside the target spaces SP. The outdoor unit mainly includes a compressor, a flow path switching valve, an outdoor heat exchanger, an outdoor expansion valve, an outdoor fan, an outdoor control unit, and various sensors such as an outdoor temperature sensor. The compressor sucks a low-pressure refrigerant, compresses the refrigerant by a compression mechanism, and discharges the compressed refrigerant to circulate the refrigerant in the refrigerant circuit. The flow path switching valve is a mechanism that switches a flow path of the refrigerant between various operating modes including a cooling operation and a heating operation. The outdoor heat exchanger causes a refrigerant flowing through the outdoor heat exchanger to exchange heat with air outside the target space SP. The outdoor expansion valve is a mechanism that adjusts a pressure and a flow rate of the refrigerant flowing in the refrigerant circuit. The outdoor fan is a fan that supplies air outside the target space SP to the outdoor heat exchanger. The outdoor control unit is configured to control the operation of each component constituting the outdoor unit. The outdoor control unit includes a control calculation device and a storage device.

The controller includes an indoor control unit and an outdoor control unit. The controller controls the operation of the entire air conditioner 50 by causing each control calculation device of the indoor control unit and the outdoor control unit to execute the program stored in each storage device. In the present embodiment, the controller performs air conditioning of the target space SP particularly based on control information received from the prediction device 10.

### (3) Processing

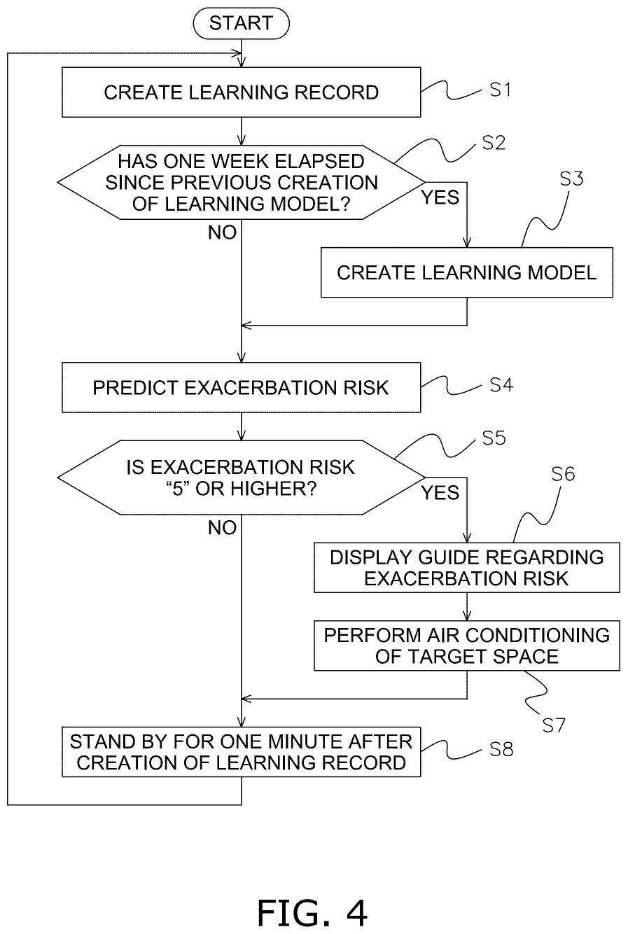

An example of processing of the exacerbation risk prediction system 1 will be described with reference to a flowchart in FIG. 4.

As a premise, it is assumed that the exacerbation risk prediction system 1 has created a learning model M in the past.

As illustrated in step S1, the prediction device 10 creates a learning record. The biological information D1, the patient information D2, and the environment information D3 of the learning record are the biological information D1, the patient information D2, and the environment information D3 acquired by the acquisition unit 191 immediately before.

When step S1 ends, as described in step S2, the prediction device 10 determines whether one week has elapsed since the previous creation of the learning model M. In a case where one week has elapsed since the previous creation of the learning model M, the processing proceeds to step S3. In a case where one week has not elapsed since the previous creation of the learning model M, the processing proceeds to step S4.

When the processing proceeds from step S2 to step S3, as described in step S3, the prediction device 10 creates the learning model M by using the learning data set created after the previous creation of the learning model M.

When the processing proceeds from step S2 to step S4 or when step S3 ends, as illustrated in step S4, the prediction device 10 predicts the exacerbation risk by inputting the biological information D1, the patient information D2, and the environment information D3 of the created learning record to the learning model M.

When step S4 ends, as illustrated in step S5, the prediction device 10 determines whether the predicted exacerbation risk is “5” or higher. In a case where the predicted exacerbation risk is “5” or higher, the processing proceeds to step S6. In a case where the predicted exacerbation risk is not “5” or higher, the processing proceeds to step S8.

When the processing proceeds from step S5 to step S6, the prediction device 10 transmits a guide regarding the exacerbation risk to the oxygen concentrator 20. When receiving the guide regarding the exacerbation risk from the prediction device 10, the oxygen concentrator 20 displays the guide regarding the exacerbation risk on the display unit 26 as illustrated in step S6.

When step S6 ends, the prediction device 10 transmits the control information to the air conditioner 50 so that the exacerbation risk next predicted becomes smaller than “0.5”. When receiving the control information from the prediction device 10, the air conditioner 50 performs air conditioning of the target space SP based on the control information as illustrated in step S7.

When the processing proceeds from step S5 to step S8 or when step S7 ends, as illustrated in step S8, the prediction device 10 stands by for one minute after the creation of the learning record.

When step S8 ends, as illustrated in step S1, the prediction device 10 creates a learning record again.

### (4) Characteristics

(4-1)

A patient suffering from a respiratory disease such as COPD is hospitalized when exacerbation occurs, and medical costs increase. In order to suppress the exacerbation, it is necessary to predict an exacerbation risk and to take measures in advance so that the exacerbation does not occur. As a conventional technique, a technique of predicting an exacerbation risk based on biological information of a patient is disclosed.

In the conventional technique, the exacerbation risk is predicted based on the biological information at rest including sleep, and the biological information on exertion is not considered. Therefore, there is a problem that the exacerbation risk cannot be sufficiently predicted.

The exacerbation risk prediction system 1 according to the present embodiment predicts an exacerbation risk of the patient P suffering from a respiratory disease. The exacerbation risk prediction system 1 includes the acquisition unit 191, the storage 11, and the prediction unit 193. The acquisition unit 191 acquires the first information including the biological information D1 and the patient information D2. The biological information D1 is information of the patient P on exertion and at rest. The patient information D2 is information of the patient P regarding a disease state. The storage 11 stores the first information. The prediction unit 193 predicts the exacerbation risk based on the first information.

In the exacerbation risk prediction system 1, the acquisition unit 191 acquires the first information including the biological information D1 and the patient information D2. The biological information D1 is information of the patient P on exertion and at rest. The prediction unit 193 predicts the exacerbation risk based on the first information. As a result, the exacerbation risk prediction system 1 can predict the exacerbation risk based on the biological information D1 of the patient P on exertion and at rest.

(4-2)

In the exacerbation risk prediction system 1 according to the present embodiment, the first information further includes the environment information D3. The environment information D3 is information of the indoor and/or outdoor of the building BL in which the patient P is present.

As a result, the exacerbation risk prediction system 1 can predict the exacerbation risk in consideration of the environment information D3.

(4-3)

In the exacerbation risk prediction system 1 according to the present embodiment, the biological information D1 is at least one of a respiratory rate, a respiratory waveform, an exhaled gas component amount, a blood oxygen concentration, a heart rate, a height, or a weight.

(4-4)

In the exacerbation risk prediction system 1 according to the present embodiment, the patient information D2 is at least one of a degree of progress of the disease state, a forced expiratory volume versus standard, a prescribed flow rate, a medical history, the type of medicine being taken, a degree of cough, a degree of sputum, a color of sputum, a degree of shortness of breath, a degree of sleep, or a physical condition.

(4-5)

In the exacerbation risk prediction system 1 according to the present embodiment, the environment information D3 of the indoor (the indoor environment information D31) is at least one of temperature, humidity, carbon dioxide concentration, carbon monoxide concentration, ozone concentration, SO3 concentration, temperature difference from the outdoor, dust amount, PM2.5 amount, yellow sand amount, mold amount, virus amount, VOC amount, pollen amount, allergic substance amount, bacteria amount, oxygen concentration, airflow, or atmospheric pressure. The environment information D3 of the outdoor (the outdoor environment information D32) is at least one of temperature, humidity, weather, atmospheric pressure, dust amount, PM2.5 amount, or yellow sand amount.

(4-6)

The exacerbation risk prediction system 1 according to the present embodiment further includes the learning unit 192. The learning unit 192 learns the first information and the evaluation regarding exacerbation in association with each other. The prediction unit 193 predicts the exacerbation risk by inputting the first information to the learning model M created by the learning unit 192.

As a result, the exacerbation risk prediction system 1 can predict the exacerbation risk by using the learning model M.

(4-7)

The exacerbation risk prediction system 1 according to the present embodiment further includes the presentation unit 194. The presentation unit 194 presents a guide regarding the exacerbation risk to the patient P based on the exacerbation risk predicted by the prediction unit 193.

As a result, the exacerbation risk prediction system 1 can notify the patient P of the exacerbation risk when the exacerbation risk is relatively high.

(4-8)

The exacerbation risk prediction system 1 according to the present embodiment further includes the operation unit 195. The operation unit 195 operates the air conditioner 50 installed in the building BL so as to improve the exacerbation risk based on the exacerbation risk predicted by the prediction unit 193.

### (5) Modifications

### (5-1) Modification 1A

In the present embodiment, the oxygen concentrator 20 is a stationary device. Alternatively, the oxygen concentrator 20 may be a portable device.

### (5-2) Modification 1B

In the present embodiment, the prediction device 10 acquires the indoor environment information D31 from the indoor environment sensor SR1. Alternatively, the prediction device 10 may acquire the indoor environment information D31 from various sensors installed in the indoor unit 51 of the air conditioner 50.

### (5-3) Modification 1C

In the present embodiment, the prediction device 10 acquires the outdoor environment information D32 from an external server of the Japan Meteorological Agency or the like. Alternatively, the prediction device 10 may acquire the outdoor environment information D32 from an outdoor environment sensor installed outside the building BL or various sensors installed in the outdoor unit of the air conditioner 50.

### (5-4) Modification 1D

In the present embodiment, the first information includes the biological information D1, the patient information D2, and the environment information D3. Alternatively, the first information may further include activity information of the patient P.

The activity information is, for example, an exertion status, an activity amount, or the like. The exertion status takes, for example, a binary value of “exertion” or “rest”. The activity amount is, for example, an amount represented by “METs” which is a unit of intensity of exercise or physical activity. The acquisition unit 191 acquires the exertion status and the activity amount from, for example, a pulse oximeter or a smart watch worn by the patient P every minute.

### (5-5) Modification 1E

In the present embodiment, the prediction device 10 acquires the first information regarding one patient P to create the learning model M. Alternatively, the prediction device 10 may acquire the first information regarding a plurality of patients and create a learning data set obtained by accumulating the first information to create the learning model M. As a result, the prediction device 10 can create a learning model in consideration of information of a plurality of patients.

### (5-6) Modification 1F

In the present embodiment, as the biological information D1, the patient information D2, and the environment information D3 of the newly created learning record, the prediction device 10 uses the biological information D1, the patient information D2, and the environment information D3 acquired by the acquisition unit 191 immediately before. However, the prediction device 10 may use not only the biological information D1, the patient information D2, and the environment information D3 acquired immediately before but also the biological information D1, the patient information D2, and the environment information D3 acquired in the past a plurality of times as the biological information D1, the patient information D2, and the environment information D3 of the learning record. For example, the prediction device 10 may include not only one “respiratory rate” acquired immediately before (as in the present embodiment) but also “respiratory rates” acquired a plurality of times in the past in the learning record as explanatory variables. As a result, the prediction device 10 can predict the exacerbation risk with higher accuracy.

(5-7)

The embodiments of the present disclosure have been described above. It will be understood that various changes to modes and details can be made without departing from the gist and scope of the present disclosure recited in the claims.

1. An exacerbation risk prediction system that predicts an exacerbation risk of a patient suffering from a respiratory disease, the exacerbation risk prediction system comprising:
an oxygen concentrator that supplies oxygen to the patient through a cannula;
an acquisition unit that acquires
first information including biological information of the patient wearing the cannula on exertion and
at rest and patient information that is information of the patient regarding a disease state;
a storage that stores the first information;
a prediction unit that predicts the exacerbation risk based on the first information; and
a learning unit that learns the first information and an evaluation regarding exacerbation in association with each other,
the biological information being at least one of a respiratory rate, a respiratory waveform, and an exhaled gas component amount,
the acquisition unit acquiring the at least one of the respiratory rate, the respiratory waveform, and the exhaled gas component amount from the oxygen concentrator, and
the prediction unit predicting the exacerbation risk by inputting the first information to a learning model created by the learning unit.

2. The exacerbation risk prediction system according to claim 1, wherein
the first information further includes environment information of at least one of an indoor and an outdoor of a building in which the patient is present.

3. The exacerbation risk prediction system according to claim 2, wherein
the biological information is at least one of a blood oxygen concentration, a heart rate, a height, and a weight.

4. The exacerbation risk prediction system according to claim 2, wherein
the patient information is at least one of a degree of progress of the disease state, a forced expiratory volume versus standard, a prescribed flow rate, a medical history, a type of medicine being taken, a degree of cough, a degree of sputum, a color of sputum, a degree of shortness of breath, a degree of sleep, and a physical condition.

5. The exacerbation risk prediction system according to claim 2, wherein
the environment information of the indoor is at least one of temperature, humidity, carbon dioxide concentration, carbon monoxide concentration, ozone concentration, SO3 concentration, temperature difference from the outdoor, dust amount, PM2.5 amount, yellow sand amount, mold amount, virus amount, VOC amount, pollen amount, allergic substance amount, bacteria amount, oxygen concentration, airflow, and atmospheric pressure, and
the environment information of the outdoor is at least one of temperature, humidity, weather, atmospheric pressure, dust amount, PM2.5 amount, and yellow sand amount.

6. The exacerbation risk prediction system according to claim 2, further comprising:
a presentation unit that presents a guide regarding the exacerbation risk to the patient based on the exacerbation risk predicted by the prediction unit.

7. The exacerbation risk prediction system according to claim 2, further comprising:
an operation unit that operates an air conditioner installed in the building so as to improve the exacerbation risk based on the exacerbation risk predicted by the prediction unit.

8. The exacerbation risk prediction system according to claim 1, wherein
the biological information is at least one of a blood oxygen concentration, a heart rate, a height, and a weight.

9. The exacerbation risk prediction system according to claim 8, wherein
the patient information is at least one of a degree of progress of the disease state, a forced expiratory volume versus standard, a prescribed flow rate, a medical history, a type of medicine being taken, a degree of cough, a degree of sputum, a color of sputum, a degree of shortness of breath, a degree of sleep, and a physical condition.

10. The exacerbation risk prediction system according to claim 8, further comprising:
a presentation unit that presents a guide regarding the exacerbation risk to the patient based on the exacerbation risk predicted by the prediction unit.

11. The exacerbation risk prediction system according to claim 1, wherein
the patient information is at least one of a degree of progress of the disease state, a forced expiratory volume versus standard, a prescribed flow rate, a medical history, a type of medicine being taken, a degree of cough, a degree of sputum, a color of sputum, a degree of shortness of breath, a degree of sleep, and a physical condition.

12. The exacerbation risk prediction system according to claim 11, further comprising:
a presentation unit that presents a guide regarding the exacerbation risk to the patient based on the exacerbation risk predicted by the prediction unit.

13. The exacerbation risk prediction system according to claim 1, further comprising:
a presentation unit that presents a guide regarding the exacerbation risk to the patient based on the exacerbation risk predicted by the prediction unit.

14. The exacerbation risk prediction system according to claim 5, further comprising:
a presentation unit that presents a guide regarding the exacerbation risk to the patient based on the exacerbation risk predicted by the prediction unit.
